(19) United States
(12) Patent Application Publication
(10) Pub. No.: US 2002/0031839 A1
(43) Pub. Date: Mar. 14, 2002

(54) METHOD AND APPARATUS FOR RAPID ANALYSIS OF ANALYTES IN BIOLOGICAL SAMPLES

(76) Inventors: JOHN C. MCNEIRNEY, FAIRBURN, GA (US); WILLIAM H. BURNS JR., ORCHARD PARK, NY (US); WILLIAM S. GIBBONS JR., EAST AURORA, NY (US)

Correspondence Address: HODGSON RUSS ANDREWS, WOODS & GOODYEAR, INTELLECTUAL PROPERTY PRACTICE GROUP, 1800 ONE M & T PLAZA, BUFFALO, NY, 14203

(21) Appl. No.: 09/208,648

(22) Filed: Dec. 10, 1998

Related U.S. Application Data
(63) Continuation of application No. PCT/US98/17758, filed on Aug. 27, 1998.
(60) Provisional application No. 60/057,192, filed on Aug. 29, 1997.

(51) Int. Cl. G01N 33/543

(52) U.S. Cl. 436/518

(57) ABSTRACT

The device and method of the present invention relate to detecting physiological changes in humans and other mammals by monitoring and detecting changes in concentration of various blood components. In particular, a concentration of an analyte is determined by immobilizing the analyte in a medium with a first antibody having a specific affinity for the analyte, labeling the analyte with a detectable second antibody, and utilizing spectrophotometric, calorimetric and fluorimetric methods of analysis to calculate the concentration.

### CROSS-REFERENCE TO A RELATED APPLICATION

Applicants hereby claim priority on earlier filed provisional patent application Ser. No. 60/057,192, filed Aug. 29, 1997, which is incorporated herein by reference.

### BACKGROUND OF THE INVENTION

1. Field of the Invention

The present invention relates generally to a method and apparatus for detection and quantitation of analytes in biological samples. More specifically, the invention relates to a method and apparatus for determining blood levels of hormones including, but not limited to, luteinizing hormones (LH), estradiol, folliclestimulating hormone (FSH), thyroid-stimulating hormone (TSH), and/or progesterone. Additionally, the invention relates to detection and determination of endocrine dysfunctions in humans and other mammals.

2. Related Art

Physiological changes in humans and other mammals are often accompanied by changes in concentration of various blood components. For example, ovulation in human and other mammalian females is preceded by a surge in the plasma concentration of luteinizing hormone (LH) in the blood. Currently, the only commercially available tests used to detect this surge are urine based. Urine tests have several drawbacks. They are awkward and often messy, and, more importantly, they are not as accurate as blood tests. In order for a detectable concentration of luteinizing hormone to be accumulated in the urine, the hormone must be released by the pituitary, circulate in the blood, be sequestered in the kidneys, and finally excreted. The completion of these processes can take as long as 12 hours after the actual plasma surge for sufficient amount of LH to accumulate in the urine. Only after such time LH can be detected by these tests. Since ovulation follows the LH surge by 12-18 hours, ovulation could have already taken place by the time the user “predicts” the occurrence using the urine test. Such a delay severely diminishes the fertile window, which usually lasts only a couple of days, and eliminates the potential for these tests to be used in a contraceptive manner. In addition, these units do not display quantitative results; rather, they indicate that the plasma concentration of LH is high or low (normal), a characteristic that excludes them from use in women whose LH peak may not be as high as that of an average woman for whom the urine tests are calibrated.

The more accurate method for LH surge detection is a blood test. Theoretically, the LH level can be known nearly instantaneously, maximizing a woman's reproductive window. Currently, however, the results of a clinical blood test are not available for 12-24 hours, and the high cost (typically-$90) is prohibitive. It is, therefore, highly desirable to have a method and device that will detect plasma concentrations of luteinizing hormone and other female reproductive hormones in a more cost effective manner and yield more timely results.

Basal plasma estradiol and FSH levels are used by fertility clinics to determine the potential for in vitro fertilization (IVF) success. Basal estradiol levels are taken on the third day of the cycle, when the concentration should be at its lowest. Studies have shown that if day three estradiol was greater than 75 pg/mL, there were no successful IVF pregnancies. If estradiol was greater than 45 pg/Ml and follicle-stimulating hormone (FSH) was greater than 17 IU/L, there were also no successful IVF pregnancies. If both basal estradiol and FSH are low (less than 46 pg/Ml and 18 IU/L, respectively), then IVF can be successful 33.8% of the time. It has been observed that basal FSH and estradiol levels obtained simultaneously on day 3 of the menstrual cycle are essential tests for determining ovarian reserve in infertile patients.“The term “ovarian reserve”reflects the future capacity of the ovaries to produce viable eggs. The primary reason that FSH levels would be elevated is that the follicles are not maturing in response to hormonal stimulation by the pituitary. As a result, the pituitary secretes more FSH. Failure to respond reflects an absence of viable ova in the ovaries, and carries with it a poor prognosis for future pregnancies.

Similarly, the cause for a particular patient's infertility can be diagnosed by monitoring various hormones. Elevated basal FSH indicates exhaustion of the ovaries, and offers a poor prognosis. In other cases, however, the cause for infertility is unrelated to the functioning of the reproductive system itself. For example, a disruption in thyroid-stimulating hormone (TSH) levels can cause an otherwise healthy reproductive system to become dysfunctional. In cases where the problem can be pinpointed to a secondary source, such as thyroid dysfunction, treatment can be highly successful. A less expensive method to screen patients for thyroid function will allow physicians to screen more infertile women more often.

Another area where a more economical and efficient method of detecting and quantizing of the levels of various hormones can be successfully implemented is luteal phase defects which affect 1-3 of infertile couples, and 1/3 of women with spontaneous abortion. The luteal phase is the time in a normal menstrual cycle after the ovum has ruptured, but preceding menses. Insufficient production of estradiol, progesterone, and/or LH during this time will prevent the endometrium and/or ovum from developing adequately, making implantation impossible. If a physician determines that the ovaries respond well enough (i.e., that there are viable eggs left), then other endocrine problems, such as luteal phase defects, can be controlled via appropriate medications. A more cost-effective method of screening patients for endocrine problems will, therefore, allow more pregnancies to be saved. In any of the above hormone assays, high costs and problematic methodology requires an infertile or amenorrheic woman to undergo many batteries of hormone tests, often with samples taken on several successive days. Therefore, a need exists to provide an effective and inexpensive device and method to allow a user to obtain prompt and reliable information of a particular hormonal state of the user.

### SUMMARY OF THE INVENTION

Accordingly, it is an object of the present invention to provide a method of rapid analysis of the blood levels of hormones including, but not limited to, luteinizing hormone, estradiol, follicle-stimulating hormone, thyroid-stimulating hormone, and/or progesterone, to predict certain physiological changes. Examples of such physiological changes include, but are not limited to, determination of ovarian state and proper function of the reproductive system, as well as detection of endocrine causality of infertility in human and other mammalian females.

It is a further object of the present invention to provide a test device for rapid detection of analytes in a biological sample. In particular, the device of the present invention serves to determine the blood level of various hormones, such as, for example, estradiol, follicle-stimulating hormone, thyroid-stimulating hormone, and/or progesterone, of a patient, and for providing the results to a user. The device of the present invention may be operated by an unskilled user and requires a minimum number of actions by the user to obtain a dependable analytical result.

A medium, such as a test strip, has an analytical matrix portion containing a porous matrix on which is immobilized a first antibody having a specific affinity for the analyte. Upon application of a biological sample to the test strip, the immobilized first antibody molecules capture the analyte. Following removal of unbound materials, detectable second antibody (label or tag) molecules having a specific affinity for the analyte are applied to the strip. Following removal of unbound materials, the amount of label immobilized on the strip is detected by a monitor and is indicative of the presence of the analyte in the sample.

Practically, the reagent impregnated test strip comprises one or more test matrix portions to which a sample is applied and then allowed to permeate through the strip material and progress into or through the strip material and into or through a detection zone in the test strip. In particular, the invention comprises a specific ELISA monoclonal antibody based assay for detecting hormones such as luteinizing hormone, estradiol, follicle-stimulating hormone, thyroid-stimulating hormone, and/or progesterone, either individually or in combination. A drop or drops of whole blood is applied to the active matrix either through a filter or directly. The analyte in the blood, as a particular hormone, for example, reacts with and binds to the primary antibody contained in a porous matrix. The analyte is then washed with a secondary antibody linked to a label, such as, for example, chromophore, rinsed again, and the extent of the resulting color development, which is indicative of the amount of the hormone in the sample, is then measured. The chromophore may be replaced by various fluorescent or luminescent tags, wherein the intensity of fluorescence or luminescence can be measured as an indicator of the amount of the analyte in the blood sample. Tests of multiple hormones are performed using the same methodology, but employing multiple active sites on the strip, wherein color development at a given site is indicative of the presence of a particular hormone. Alternatively, a single active matrix impregnated with multiple primary antibodies having specific affinity to specific hormones serves as a binding site for such hormones, wherein chromophore, fluorescence or luminescence tags for different secondary antibodies bound to respective hormones are then utilized to detect and quantize the levels of hormones in the blood sample.

Another embodiment of the invention consists of a single or multiple cell liquid phase assay, in which a whole blood sample is collected and applied either manually or automatically to one or more cells, and specific ELISA monoclonal antibody based assay for luteinizing hormone, estradiol, follicle-stimulating hormone, thyroid-stimulating hormone, and/or progesterone, either individually or in combination, is performed. In this embodiment the hormones react with the primary antibody contained in a porous matrix, washed with a secondary antibody linked to a chromophore, rinsed, and the extent of the resulting color development, which is proportional to the amount of hormones in the same, is then measured. Again, the chromophore may be replaced by fluorescent or luminescent tags, wherein the intensity of fluorescence or luminescence is measured. Tests of multiple hormones can be performed in a single cell or multiple cells.

If a chromophore is used, the developed color can be measured by visual comparison to a color chart but is preferably measured spectrophotometrically, because visual comparison to a color chart is less accurate and provides only a rough (+/−15%) approximation of the LH level. The intensity of fluorescence and luminescence is preferably determined spectrophotometrically.

Still other objects and advantages of the present invention will become readily apparent to those skilled in this art from the following detailed description, wherein a preferred embodiment is shown and described, simply by way of illustration of the best mode contemplated by the inventors for carrying out the invention. As will be realized, the invention is capable of other and different embodiments, and its several details are capable of modifications in various obvious respects, all without departing from the invention. Accordingly, the drawings and description are to be regarded as illustrative in nature and not as restrictive.

### BRIEF DESCRIPTION OF THE DRAWINGS

FIG. 1A is a top view of a test strip according to the principles of the present invention.

FIG. 1B is a cross-sectional side view of a test strip.

FIG. 2 contains a side view of a liquid phase ELISA test strip according to the principles of the present invention.

FIG. 3A is a top view of a reflectance spectrophotometric device processing the test strips of FIGS. 1A-1B and FIG. 2.

FIG. 3B is a side view of a reflectance spectrophotometric device of FIG. 3A.

FIG. 4 is a schematic diagram of a device for processing the solid phase test strip of FIGS. 1A-1B.

FIG. 5 is a schematic diagram of a device for processing the liquid phase test strip of FIG. 2.

### DETAILED DESCRIPTION OF THE INVENTION

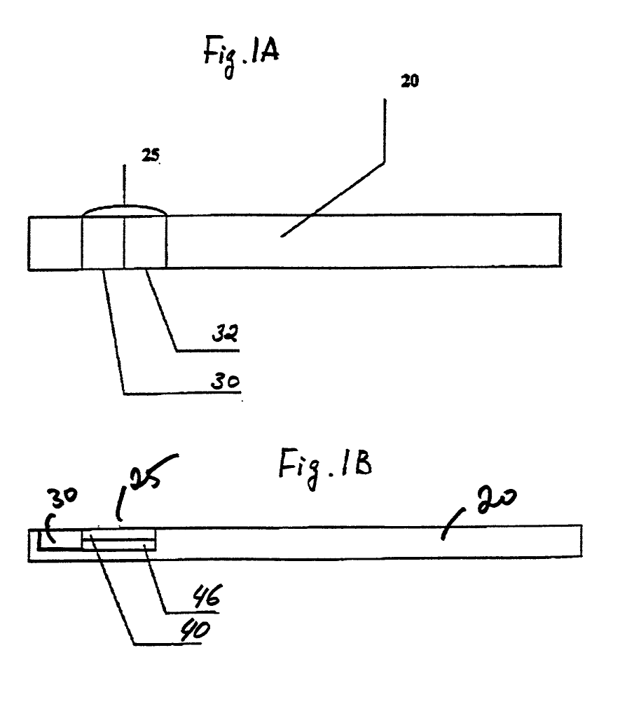

Shown in FIG. 1A is a solid phase test strip 20 which comprises an entire analytical matrix 25 having two zones: a first zone 30 serving the function of a “blank” and a second zone 32. The term “blank” is understood to have the meaning commonly known in the calorimetric and photometric analytical arts.

Second zone 32 is the reactant matrix zone. Zone 32 contains a first antibody in a porous nonreactive carrier matrix 46. Such matrices are commonly used in the art for nucleic acid and protein binding. Examples of materials for a suitable carrier matrix include nitrocellulose and nylon. When carrier matrix 46 is nitrocellulose, antibodies can be directly immobilized on to the carrier matrix without the need of a chemical treatment. However, for other matrices, immobilization can be accomplished by techniques well known in the art such as treatment with cyanogen bromide, and carbonyldiimidazole.

The first antibody is selected from the group consisting of a monoclonal antibody, a polyclonal antibody,and fragments thereof. In a preferred embodiment, the first antibody molecule is a monoclonal antibody. The particular choice of antibody will depend upon the analyte to be detected. For example for the detection of luteinizing hormone (LH), specific antibodies to LH can be immobilized on to carrier matrix 46.

For the purpose of removing unbound materials from carrier matrix 46, a washing agent is provided in the form of wet absorbent wipe. The wipe is preferably made of a non-woven material. The wipe is wet with an appropriate solution. The wipes may be wet beforehand or may be wetted at the time of the analysis. For example, for blocking purposes, wipes may be wet with a buffer containing well known blocking agents. Suitable buffers include phosphate, tris, glycine and the like, generally in the molarity of 0.1 to 3.0. Suitable blocking agents include but are not limited to bovine serum albumin, diluted serum, non-fat dry milk, and casein. Wipes wet with the washing solution are referred to herein as washing wipes and the wipes wet with blocking solution are referred to herein as blocking wipes.

The second antibody also has an affinity for the analyte, preferably for a different epitope than the first antibody. The second antibody is selected from the group consisting of monoclonal antibody, polyclonal antibody and fragments thereof. In a preferred embodiment, the second antibody is a monoclonal antibody directed to an epitope distinct from the epitope of the first antibody.

The second antibody is labeled with a detectable molecule or complex. Suitable detectable labels include chromophores and fluorescent molecules and complexes. Fluorescent labeled antibodies to specific analytes are available commercially or can be prepared by using techniques known in the art. Kits for fluorescent labeling of antibodies are available commercially (e.g. molecular probes or from Pierce). It is preferable to store the second antibody in a light protected compartment. The second antibody solution may be supplied as a liquid or as a wet wipe similar to washing and blocking wipes. A suitable storage compartment for a solution of labeled antibody is foil-wrapped applicator tube, or dark colored tube. A suitable storage compartment for labeled antibody solution on wet wipes is foil-wrapped packages.

To detect the presence of an analyte in a biological sample, a small amount of the sample, for example a drop of blood is applied to test strip 20 and allowed to react for a suitable period of time (about 30 seconds to several minutes). To reduce nonspecific binding, analytical matrix 25 may be wiped with a blocking wipe before application of the sample. A washing wipe is used to gently wipe off the unbound materials. Additionally, a blocking wipe may also be used to reduce non-specific binding. Following removal of unbound materials, analytical matrix 25 is wiped with a wipe containing a labeled second antibody. After suitable incubation, the analytical matrix is again wiped with the washing and/or blocking wipes and test strip 20 inserted into an analyzing device for analysis.

To give a particular example, in one embodiment of the present invention, second zone 32 contains a specific anti-human monoclonal antibody (the first antibody) or a set of antibodies bound to the second zone 32 of analytical matrix 25. The specific characteristics of each kind of antibodies depend on the analyte being tested. A sample of whole blood from a patient is obtained by a finger-prick, or other standard method. The blood drop(s) are applied by conventional means to analytical matrix 25 where the cellular matter in the blood is filtered by a removable filter 40 covering second zone 32. The serum that passes through filter 40 is allowed to react with the first antibody attached to second zone 32, and allowed to encounter first zone 30, which contains blocked substrate without antibodies. Filter 40 is then removed, and a reactive site 46 is washed to remove unreacted antigen. Then secondary antibody, labeled with a detectable complex, such as a chromophore, fluorescent, or luminescent complex specific for the first antibody or for the initial antigen, are applied. Strip 20 is then rinsed to remove uncombined secondary antibody. Since the intensity of the color, fluorescence, or luminescence is indicative of the amount of the chromophore, fluorescent, luminescent or other label immobilized on the test strip 20, therefore measuring such intensity is also indicative of the amount of hormone contained in the drop(s) of blood.

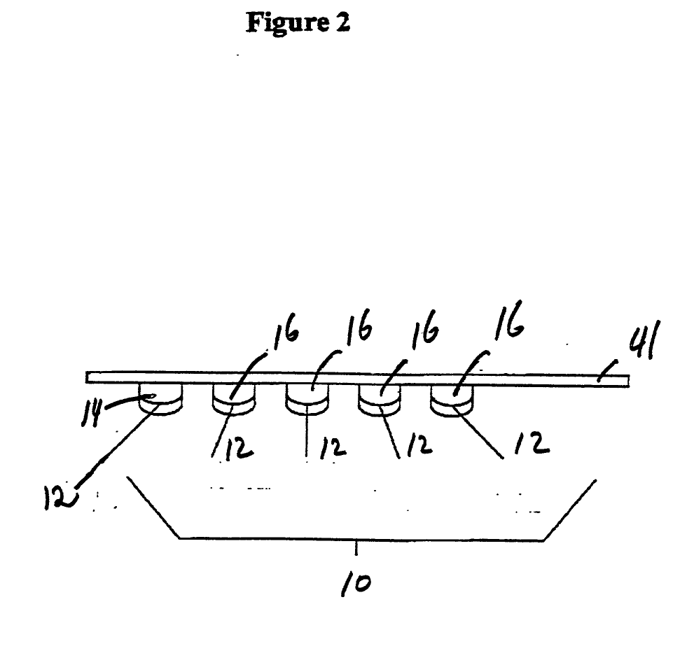

A liquid phase ELISA test plate 10 according to the present invention is shown in FIG. 2. Test plate 10 comprises liquid holding cells 12, one of which is designated as a reference or blank cell 14 and one or more other cells are designated as test cells 16. The cells 12 are covered by a removable filter 41. In one embodiment, each cell 16 contains a specific anti-human monoclonal antibody, the first antibody, bound to its walls. A sample of whole blood from a patient is obtained by a finger-prick, or other standard method. The blood drop(s) are applied by conventional means to each cell, and the cellular matter in the blood is filtered by the removable filter 41. The serum that passes through filter 41 is allowed to react with the first antibody bound to cell walls of cells 16, and allowed to encounter blank cell 14 containing blocked substrate without the first antibody. Filter 40 is then removed, and the cells are washed to remove unreacted material. A secondary antibody, specific for the conjugated first primary antibody or for the initial analyte, labeled with a chromophore, fluorescent, or luminescent complex is applied to all the cells. The cells are then rinsed to remove the uncombined secondary complex and the intensity of the color, fluorescence or luminescence is measured. The intensity of the color, fluorescence, or luminescence is indicative of the amount of the chromophore, fluorescent, or luminescent complex, respectively, present in the test strip which, in turn, is indicative of the amount of analyte in the drop(s) of blood.

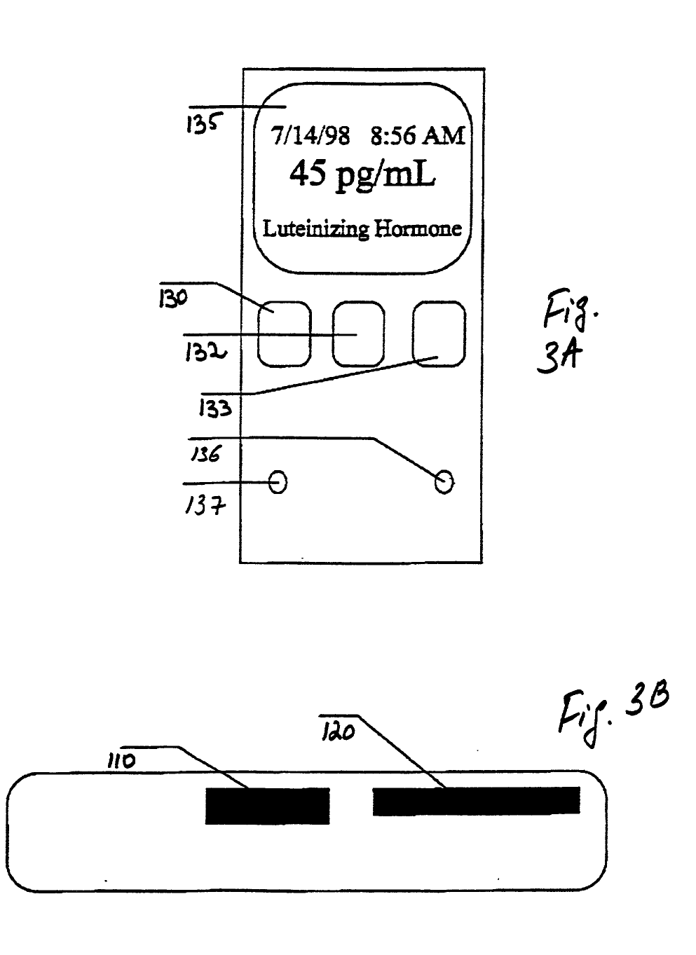

The color intensity in either embodiment of the invention may be measured by comparison to a color chart in order to determine the blood level of the desired analyte. However, the preferred device for measuring the intensity of the color, and, thus, determining the level of hormone in the sample, is a device 100, as illustrated in FIGS. 3A and 3B. Device 100 is preferably a portable, handheld reflectance spectrophotometer devised to receive and “read” the developed strip, (i.e., determine the hormone level represented by the intensity of the developed color), display the results to an operator, and record the results on a memory device.

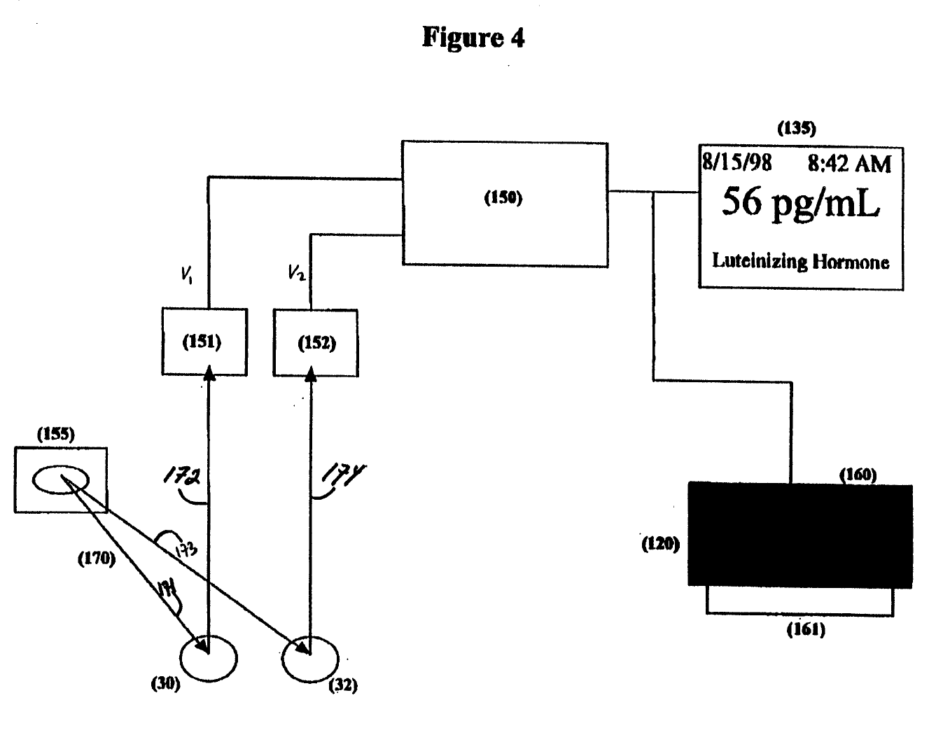

In a preferred embodiment, illustrated in FIGS. 3A-3B and 4) device 100 comprises a reflectance spectrophotometer which includes a Light Emitting Diode (LED) 155 (represented pictorially in FIG. 4 at 155) emitting a light beam 170 (shown pictorially in FIG. 4 at 170), which light beam 170 comprises a first beam 171 and a second beam 173. Device 100 can also comprise a timer 136 and a switch for turning LED 155 on and off. In a preferred embodiment of the invention light beam 170 is a monochromatic beam. When a test strip is inserted into test strip receiving means 110 of device 100, light beams 171 and 173 impinge on zones 30 and 32, respectively, of the test strip. First zone 30 reflects first light beam 171 as a reflected beam 172, second zone 32 reflects second light beam 173 as a reflected beam 174. Reflected light beams 172 and 174 are received by photodetectors 151 and 152, respectively, as shown in FIG. 4. Since first zone 30 is a blank zone with no chromophore bound to it, no detectable change of intensity of first light beam 171 will occur upon its incidence on and reflectance from first zone 30 and, thus, the intensity of reflected beam 172 detected by photodetector 151 will not be detectably different from that of first beam 171. The chromophore contained in second zone 32 will absorb at least some light from light beam 173 and, therefore, the intensity of reflected beam 174 detected by photodetector 152 will differ from that of reflected beam 172. Voltages V₁ and V₂, the outputs of photodetectors 151 and 152, respectively, are then inputted to a signal processor 150. Signal processor 150 utilizes the difference between voltages V₁ and V₂ to calculate the concentration of the analyte in the sample. In the preferred embodiment signal processor 150 subtracts voltage V₁, corresponding to the blank zone signal from voltage V₂ corresponding to the reacted zone signal and then references the difference to a standard voltage curve representing hormone levels. Signal processor 150 may include means for storing information and data as is commonly known in the art. The concentration of the analyte is displayed in a display window 135, which is a Liquid Crystal Display (LCD) in the preferred embodiment.

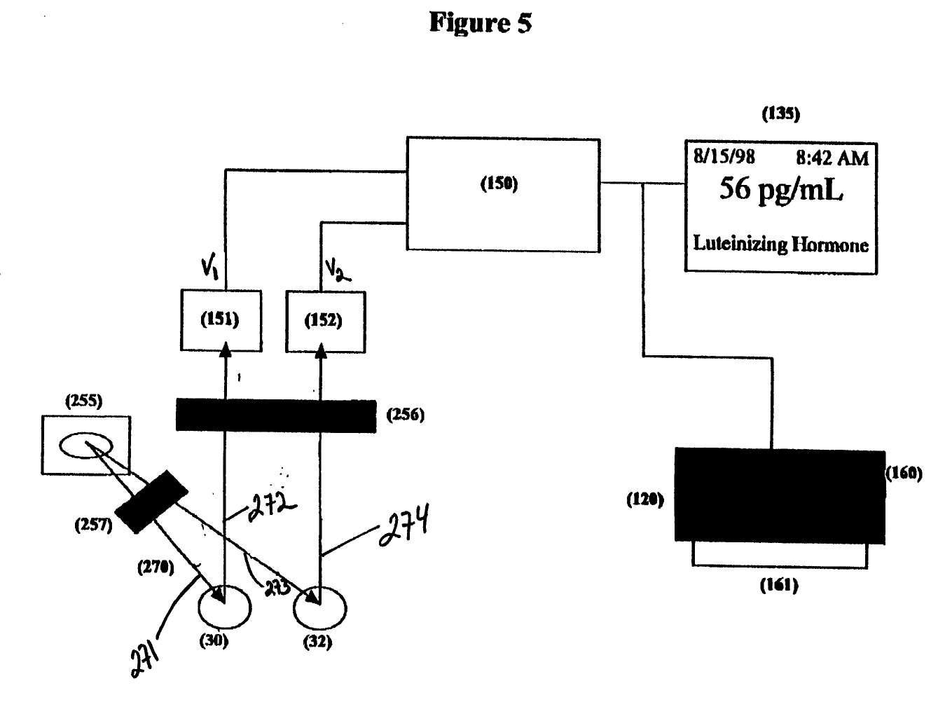

Another embodiment of the present invention, wherein the secondary antibody comprises a fluorescent/luminescent complex (tag), is illustrated pictorially in FIG. 5. A light source 255 generates a light beam 270 which can be either monochromatic or broadband light. In the preferred embodiment the light is broadband, filtered by filtering means 257 for specific wavelength light - the excitation wavelength for a given fluorescent tag. Light beam 270 comprises a first light beam 271 impinging on first zone 30 of the test strip, and a second beam 273 impinging on second zone 32 of the strip. The fluorescent tag contained in second zone 32 fluoresces in response to excitation caused by second beam 273, emitting a light beam 274 of a different (longer) wavelength. The wavelength of a light beam 272 reflected by first zone 30 is not affected by the presence of a fluorescent tag, because first zone 32 is a blank zone with no fluorescent material. The photodetectors 151 and 152 detect the light beams 272 and 274, respectively, filtered for the desired fluorescence wavelength by filtering means 256. Filters 256 and 257 are selected to correspond to a particular desired wavelength. For each sample tested by the device of the present invention appropriate filters 256 and 257 are selected and used in the device. Filtering of excitation and fluorescent emission wavelengths is performed by filtering means well known to those skilled in the art. Alternatively unfiltered emitted light can be detected by photodetectors 151 and 153, and wavelength and amplitude data determined by processor 150 using standard signal processing means well known to those in the art.

Multiple assays may be performed for either the embodiment pictured in FIGS. 1A-1B or the embodiment pictured in FIG. 2 in at least two ways: (i) by using a test strip with one active zone, wherein only one hormone can be tested per active zone or (ii) by using one or more active zones per strip, wherein multiple hormones are tested in some or all active zones. For multiple strips having the same excitation and emission wavelength, multiple sources 255 or photodetectors 152 may be required. Mobility of source 255, photodetector 152, or test strip 20 can eliminate multiplicity requirements. For testing multiple hormones in a given active matrix, multiple excitation filters 257 and/or multiple emission filters 156 can be used to sequentially test hormones having varied fluorescent tags. Alternatively, processor 150 can use signal processing means to determine frequency and wavelength composition of light detected by photodetector means. Signal processing means will be well known to those skilled in the art.

Device 100 may include a magnetic car writer (shown pictorially in FIGS. 4 and 5 at 160) for storing the output of the signal processor 150, along with date and time information, on a removable magnetic card (shown pictorially in FIGS. 4 and 5 at 161). The removable magnetic card 161 is inserted into magnetic card receiving means 120 in order to perform read and write operations. A floppy disk can be used for the same purpose with device 100.

Device 100 may include an ON/OFF switch 130, a means 132 for initializing recall and display of data stored on the magnetic card, or in the memory of the signal processor, means 133 for ejecting the magnetic card, and display means 135 for displaying analytical results and date and time information.

It is intended that the above information of preferred embodiments of the structure of the present invention and the description of its operation are but one or two enabling best mode embodiments for implementing the invention. Other modifications are variations are likely to be conceived of by those skilled in the art upon reading of the preferred embodiments and a consideration of the appended claims and drawings. These modifications and variations still fall within the breadth and scope of the disclosure of the present invention.

What is claimed is:

1. A device for detecting the presence of at least one analyte in a biological sample, the device comprising:
a source generating a light beam having one or more predetermined characteristics, the light beam being incident on the biological sample;
a detector receiving an altered light beam propagating from the biological sample to the detector, the altered light beam having one or more characteristics different from one or more predetermined characteristics of the incident light beam, the difference between the characteristics of the incident light beam and the altered light beam being caused by the presence of the biological sample; and
a processor utilizing the difference between the characteristics of the incident light beam and the altered light beam to detect the presence of at least one analyte in the biological sample.

2. The device of claim 1 further comprising a display communicating with the processor and indicating the presence of the analyte in the biological sample.

3. The device of claim 1 further comprising a memory device communicating with the processor and storing information about the presence of at least one analyte in the biological sample.

4. The device of claim 3, wherein the memory device is a removable magnetic card or a disk.

5. The device of claim 1 further comprising a first filter disposed between the source and the biological sample, the first filter serving to provide the incident light beam with one or more predetermined characteristics, and a second filter disposed between the biological sample and the detector, the second filter serving to provide the altered light beam received by the detector.

6. The device of claim 5, wherein the biological sample comprises a fluorescent substance.

7. The device of claim 6, wherein the predetermined characteristic is a wavelength of the incident light beam.

8. The device of claim 6, wherein the altered light beam has a wavelength different from that of the incident light beam.

9. The device of claim 1, wherein the biological sample comprises a chromophore.

10. The device of claim 9, wherein the predetermined characteristics are an intensity and a wavelength of the incident light beam.

11. The device of claim 9, wherein the altered light beam has an intensity different from that of the incident light beam.

12. The device of claim 1, wherein the analyte is selected from the group consisting of a luteinizing hormone, estradiol, FSH, TSH, progesterone, and combinations thereof.

13. A kit for detecting the presence of at least one analyte in a biological sample, the kit comprising: a medium comprising an analytical matrix having a reactant zone to which the biological sample is applied, the reactant zone containing immobilized first antibody having specific affinity for the analyte;
a detectable second antibody with a detectable label, the second antibody having specific affinity for the analyte; and
a device for detecting the presence of the analyte in the biological sample, the device comprising:
a source generating a light beam having one or more predetermined characteristics, the light beam being incident on the biological sample;
a detector receiving an altered light beam propagating from the biological sample to the detector, the altered light beam having one or more characteristics different from one or more predetermined characteristics of the incident light beam, the difference between the characteristics of the incident light beam and the altered light beam being caused by the presence of the biological sample; and
a processor utilizing the difference between the characteristics of the incident light beam and the altered light beam to detect the presence of at least one analyte in the biological sample.

14. The kit of claim 13, wherein the first antibody is selected from the group consisting of a monoclonal antibody, polyclonal antibody, and fragments thereof.

15. The kit of claim 13, wherein the second antibody is selected from the group consisting of a monoclonal antibody, polyclonal antibody, and fragments thereof.

16. The kit of claim 13, wherein the first antibody and the second antibody are monoclonal antibodies and wherein the first antibody and the second antibody bind to different epitopes of the analyte.

17. The kit of claim 13, wherein the detectable label on the second antibody is a fluorescent complex or molecule.

18. The kit of claim 13, wherein the detectable label on the second antibody is a chromophore.

19. The kit of claim 17, wherein the fluorescent complex or molecule is selected from the group consisting of fluorescein, rhodamine, texas red, and combinations thereof.

20. The kit of claim 13, wherein the analyte is selected from the group consisting of a luteinizing hormone, estradiol, FSH, TSH, progesterone, and combinations thereof.

21. The kit of claim 13, wherein the medium is a strip.

22. A method of detecting the presence of at least one analyte in a biological sample, the method comprising: providing a light beam incident on the biological sample and having at least one predetermined characteristic;
causing the biological sample to alter the incident light beam in such a way that the incident light beam transforms into an altered light beam propagating between the biological sample and a detector, the altered light beam having at least one characteristic different from that of the incident light beam;
detecting the altered light beam by the detector; and
utilizing the difference of at least one characteristic of the incident light beam and the altered light beam to provide an indication of the presence of at least one analyte in the biological sample.

23. A method of detecting the presence of at least one analyte in a biological sample, the method comprising: providing a medium comprising an analytical matrix having a reactant zone, the reactant zone containing immobilized first antibody having specific affinity for the analyte;
applying the biological sample to the reactant zone;
applying a detectable second antibody with a detectable label to the reactant zone, the second antibody having specific affinity for the analyte;
illuminating the reactant zone with an incident light beam having at least one predetermined characteristic;
detecting the altered light beam resulting from a change caused to at least one characteristic of the incident light beam by the detectable label; and
utilizing a difference between at least one characteristic of the incident light beam and the altered light beam to detect the presence of at least one analyte in a biological sample.

24. The method of claim 23, wherein the first antibody is selected from the group consisting of a monoclonal antibody, polyclonal antibody, and fragments thereof.

25. The method of claim 23, wherein the second antibody is selected from the group consisting of a monoclonal antibody, polyclonal antibody, and fragments thereof.

26. The method of claim 23, wherein the first antibody and the second antibody are monoclonal antibodies and wherein the first antibody and the second antibody bind to different epitopes of the analyte.

27. The method of claim 23, wherein the detectable label on the second antibody is a fluorescent complex or molecule.

28. The method of claim 23, wherein the detectable label on the second antibody is a chromophore.

29. The method of claim 27, wherein the fluorescent complex or molecule is selected from the group consisting of fluorescein, rhodamine, texas red, and combinations thereof.

30. The method of claim 23, wherein the analyte is selected from the group consisting of a luteinizing hormone, estradiol, FSH, TSH, progesterone, and combinations thereof.

31. The method of claim 23, wherein the medium is a strip.
